(19) United States
(12) Patent Application Publication
(10) Pub. No.: US 2021/0259693 A1
(43) Pub. Date: Aug. 26, 2021

(54) SURGICAL STAPLING DEVICE WITH FLEXIBLE SHAFT

(71) Applicant: Covidien LP, Mansfield, MA (US)

(72) Inventors: Srinivasa Murthy Aravalli AVVLN, Hyderabad (IN); Ravi Sekhar GUTTI, Hyderabad (IN); Rajanikanth MANDULA, Hyderabad (IN); Jitendra Bhargava SRINIVAS, Hyderabad (IN)

(21) Appl. No.: 17/113,494

(22) Filed: Dec. 7, 2020

Related U.S. Application Data
(60) Provisional application No. 62/981,633, filed on Feb. 26, 2020.

(51) Int. Cl. A61B 17/115

(52) U.S. Cl. CPC A61B 17/1155; A61B 2017/07285

(57) ABSTRACT

A surgical stapling device includes a tool assembly having an anvil assembly and a shell assembly. The shell assembly includes a housing that supports first and second motors, a staple cartridge supporting a plurality of staples, and a knife. The first motor can be energized to eject the staples from the staple cartridge and the second motor can be energized to advance the knife to cut tissue.

### CROSS-REFERENCE TO RELATED APPLICATION

This application claims the benefit of and priority to U.S. Provisional Patent Application No. 62/981,633 filed Feb. 26, 2020, the entire disclosure of which is incorporated by reference herein.

### FIELD

This disclosure is directed to stapling devices and, more particularly, to endoscopic stapling devices with flexible shafts that support an end effector.

### BACKGROUND

Surgical stapling devices for performing surgical procedures endoscopically are well known and are commonly used to reduce patient trauma and shorten patient recovery times. Typically, an endoscopic stapling device includes a handle assembly, a rigid elongate body that extends distally from the handle assembly, and an end effector including a tool assembly that is supported on a distal portion of the elongate body. The handle assembly is coupled to the end effector by drive mechanisms that extend through the elongate body such that a clinician can control operation of the end effector remotely via the handle assembly.

Surgical stapling devices for endoscopic use are available in a variety of configurations including linear and circular. Circular stapling devices are commonly used to perform end to end anastomoses after resections of the large bowel, i.e., colectomies. In a large percentage of colectomy procedures, the portion of the colon that must be resected is in the ascending colon or the transverse colon which cannot be easily accessed by a circular stapling device having a rigid shaft. As such, these procedures are typically performed during an open colectomy procedure which result in increased patient trauma and recovery time.

A continuing need exists in the medical arts for a stapling device of minimal complexity having a flexible shaft for accessing a surgical site.

### SUMMARY

One aspect of this disclosure is directed to a surgical stapling device including a handle assembly, an elongate body, and a tool assembly. The elongate body includes a flexible outer tube. The tool assembly includes an anvil assembly and a reload assembly including a housing, a staple cartridge, a plurality of staples, a staple pushing member, a knife, a first motor, and a second motor. The staple cartridge defines a plurality of staple pockets. Each of the plurality of staples is supported within one of the plurality of staple pockets. The staple pushing member is positioned within the housing and is movable from a retracted position to an advanced position to eject the plurality of staples from the staple cartridge. The knife is supported within the housing and is movable from a retracted position to an advanced position to cut tissue. The first and second motors are supported within the housing and can be energized to move the staple pushing member and the knife, respectively, from their retracted positions to their advanced positions.

Another aspect of the disclosure is directed to a reload assembly including a housing, a staple cartridge, a plurality of staples, a staple pushing member, a knife, a first motor, and a second motor. The staple cartridge defines a plurality of staple pockets. Each of the plurality of staples is supported within one of the plurality of staple pockets. The staple pushing member is positioned within the housing and is movable from a retracted position to an advanced position to eject the plurality of staples from the staple cartridge. The knife is supported within the housing and is movable from a retracted position to an advanced position to cut tissue. The first and second motors are supported within the housing and can be energized to move the staple pushing member and the knife, respectively, from their retracted positions to their advanced positions.

Yet another aspect of the disclosure is directed to a surgical stapling device including an elongate body and a tool assembly. The tool assembly includes an anvil assembly, and a reload assembly. The reload assembly includes a housing, a staple cartridge, a plurality of staples, a staple pushing member, a knife, a first motor, and a second motor. The staple cartridge defines a plurality of staple pockets. Each of the plurality of staples is supported within one of the plurality of staple pockets. The staple pushing member and the knife are positioned within the housing and are movable from retracted positions to advanced positions to eject the plurality of staples from the staple cartridge and to cut tissue, respectively. The first and second motors are supported within the housing and can be energized to move the staple pushing member and the knife from their retracted positions to their advanced positions.

In aspects of the disclosure, the first and second motors are piezo electric transducers.

In some aspects of the disclosure, the first and second motors are coupled to actuation buttons in the handle assembly by wires.

In certain aspects of the disclosure, the handle assembly supports a battery pack.

In aspects of the disclosure, the anvil assembly includes an anvil head and an anvil center rod, wherein the anvil head supports an anvil plate.

In some aspects of the disclosure, the staple cartridge and the anvil plate have an annular configuration.

In certain aspects of the disclosure, the stapling device includes an approximation assembly having an anvil retainer that is releasably coupled to the anvil assembly and movable from an advanced position to a retracted position to move the tool assembly from an open position to a clamped position.

In aspects of the disclosure, the approximation assembly extends from the handle assembly through the elongate body to the tool assembly.

In some aspects of the disclosure, the stapling device includes a knife carrier supported within the housing and supporting the knife, wherein the knife carrier is movable from a retracted position to an advanced position to move the knife from its retracted position to its advanced position.

In aspects of the disclosure, the knife carrier is coupled to the second motor such that when the second motor is deenergized, the knife carrier and the knife are returned to their retracted positions.

In certain aspects of the disclosure, the reload assembly includes an annular pusher supported within the housing that is in abutting relation with the staple pushing member and movable from a retracted position to an advanced position in response to energization of the first motor to move the staple pushing member from its retracted position to its advanced position.

In aspects of the disclosure, the annular pusher is coupled to the second motor such that when the second motor is deenergized, the annular pusher is returned to its retracted positions.

Other aspects of the disclosure will be appreciated from the following description.

### BRIEF DESCRIPTION OF THE DRAWINGS

Various exemplary aspects of the disclosed surgical stapling device are described herein below with reference to the drawings, wherein:

FIG. 1 is a side perspective view of a surgical stapling device including exemplary aspects of the disclosure with a tool assembly in an open position;

FIG. 2 is an exploded view of the tool assembly of the stapling device shown in FIG. 1;

FIG. 3 is a cross-sectional view taken along section line 3-3 of FIG. 1 with the tool assembly moved to a clamped position prior to firing of the tool assembly;

FIG. 4 is a side partial cross-sectional view of the stapling device shown in FIG. 1 positioned within the colon of a patient;

FIG. 5 is a cross-sectional view taken along section line 3-3 of FIG. 1 with the tool assembly in the clamped position as the tool assembly is actuated to fire staples from the tool assembly;

FIG. 6 is a cross-sectional view taken along section line 3-3 of FIG. 1 with the tool assembly in as the tool assembly is actuated to advance the knife assembly to cut tissue;

FIG. 7 is a perspective view from the distal end of an exemplary aspect of the knife assembly of the tool assembly of the stapling device shown in FIG. 1; and

FIG. 8 is a perspective view from the distal end of another exemplary aspect of the knife assembly of the tool assembly of the stapling device shown in FIG. 1.

### DETAILED DESCRIPTION

The disclosed surgical stapling device will now be described in detail with reference to the drawings in which like reference numerals designate identical or corresponding elements in each of the several views. However, it is to be understood that the aspects of the disclosure described herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “endoscopic” is used generally to refer to endoscopic, laparoscopic, arthroscopic, and/or any other procedure conducted through small diameter incision or cannula. Further, the term “clinician” is used generally to refer to medical personnel including doctors, nurses, and support personnel.

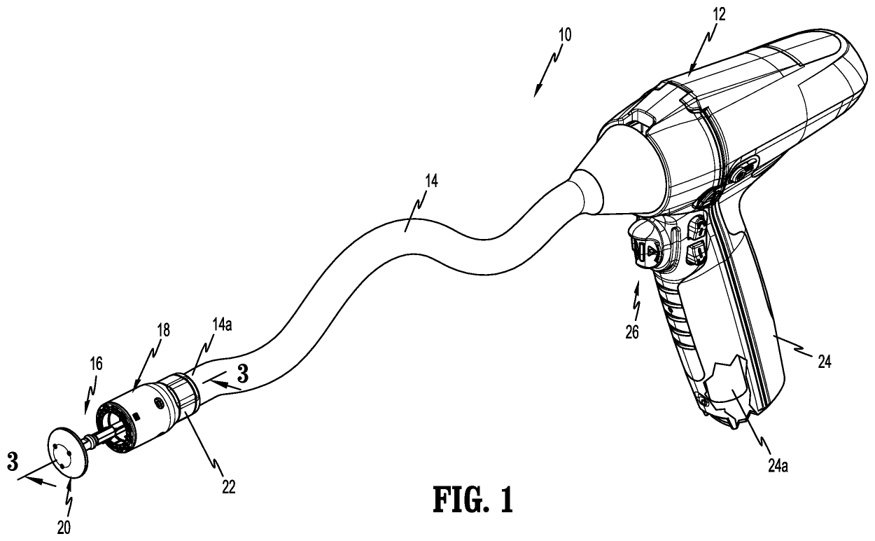

FIG. 1 illustrates a circular stapling device shown generally as stapling device 10 including exemplary aspects of the disclosure. The stapling device 10 includes a handle assembly 12, an elongate body 14, and a tool assembly 16. The tool assembly 16 includes a reload or shell assembly 18, and an anvil assembly 20 that is supported for movement in relation to the reload assembly 18 between open and clamped positions. In aspects of the disclosure, the reload assembly 18 includes a proximal coupling portion 22 that is releasably coupled to a distal portion 14a of the elongate body 14 to facilitate replacement of the reload assembly 18 and reuse of the stapling device 10. Alternately, the reload assembly 18 can be fixedly secured to the elongate body 14. The handle assembly 12 includes a stationary grip 24 that supports actuation buttons 26 for controlling operation of various functions of the stapling device 10 including approximation of the reload and anvil assemblies 18, 20, firing of staples from the reload assembly 18, and cutting or coring of tissue.

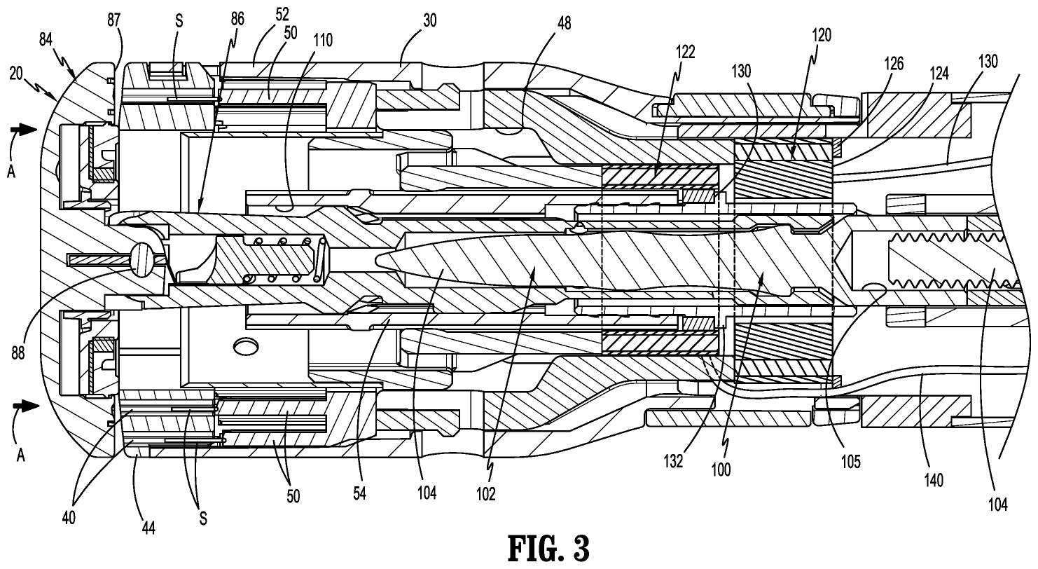

The stapling device 10 is an electrically powered stapling device. As such, the handle assembly 12 may support a battery or battery pack 24a. The elongate body 14 includes an approximation mechanism 100 (FIG. 3) that translates power from the handle assembly 12 to the anvil assemblies 20 to facilitate movement of the tool assembly 16 from the open position (FIG. 1) to the clamped position (FIG. 3) as described in further detail below. Examples of electrically powered stapling devices can be found in U.S. Pat. No. 9,055,943 (the '943 Patent), U.S. Pat. No. 9,023,014 (the '014 Patent), and U.S. Publication Nos. 2018/0125495, and 2017/0340351.

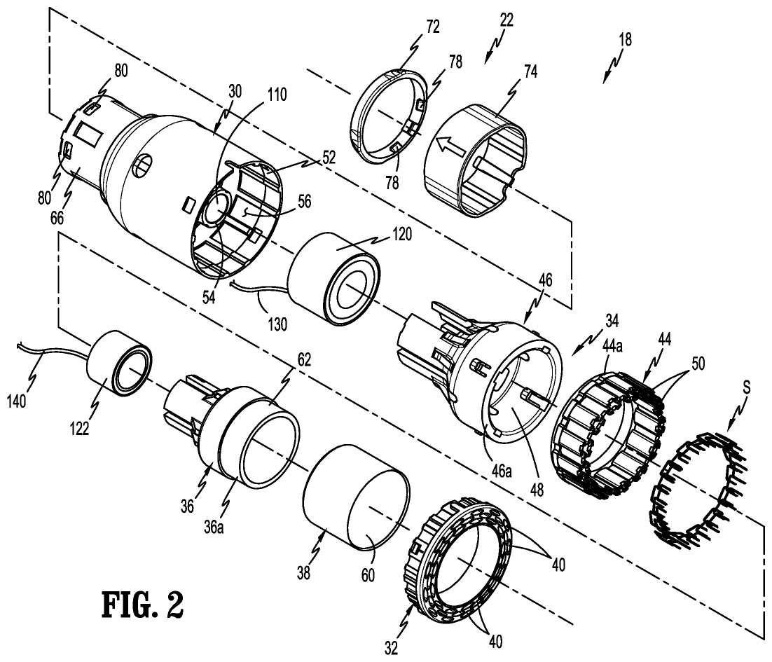

FIG. 2 illustrates an exploded view of the reload assembly 18 of the stapling device 10 shown in FIG. 1. The reload assembly 18 includes a housing 30, a staple cartridge 32, a plurality of staples “S”, a staple pusher assembly 34, a knife carrier 36, and an annular knife 38. The staple cartridge 32 is annular and defines annular rows of staple pockets 40. Each of the staple pockets 40 supports one of the plurality of staples “S” (FIG. 3).

The pusher assembly 34 of the reload assembly 18 includes a staple pushing member 44 and an annular pusher 46 that together define a longitudinal through bore 48 (FIG. 3). The annular pusher 46 has a distal portion 46a that abuts a proximal portion 44a of the staple pushing member 44 such that distal movement of the annular pusher 46 within the housing 30 of the reload assembly 18 causes distal movement of the staple pushing member 44 within the housing 30. The staple pushing member 44 of the reload assembly 18 has a plurality of distally extending fingers 50. Each of the fingers 50 is received within a respective one of the staple pockets 40 of the staple cartridge 32 and is movable through the respective staple pocket 40 to eject the staples “S” from the staple pockets 40 when the staple pushing member 44 is moved from a retracted position to an advanced position within the housing 30. In some aspects of the disclosure, the staple cartridge 32 is annular and includes a plurality of annular rows of staple pockets 40, e.g., 2, 3, etc.

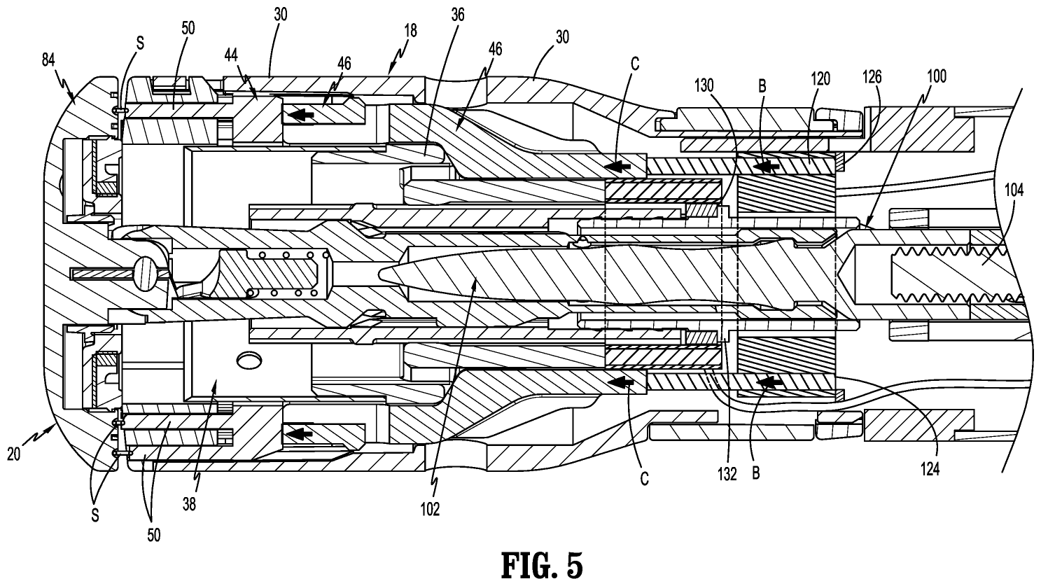

The shell housing 30 of the reload assembly 18 includes an outer housing portion 52 and an inner housing portion 54 that is spaced from the outer housing portion 52 to define an annular cavity 56 between the outer and inner housing portions 52, 54. The pusher assembly 34 and the knife carrier 36, including the annular knife 38, are supported within the annular cavity 56 and movable between retracted (FIG. 3) and advanced positions (FIG. 5). The pusher assembly 34 is movable from its retracted position to its advanced position (FIG. 5) independently of the knife carrier 36 to eject the staples “S” from the staple cartridge 32.

The annular knife 38 is supported about an outer surface of the knife carrier 36 and defines a cylindrical cavity 60. In aspects of the disclosure, the knife carrier 36 includes an outer surface 36a that defines a shoulder 62 that supports a proximal end of the annular knife 38. The annular knife can be press fit onto the knife carrier 36. Alternately, other fastening techniques may be used to secure the annular knife 38 to the knife carrier 36.

The shell housing 30 includes a proximal portion 66 that supports the coupling mechanism 22 which is operable to couple the reload assembly 18 to the elongate body 14 (FIG. 1) of the stapling device 10. The coupling mechanism 22 includes a retaining member 72 and a coupling member 74. The coupling member 74 is received about the proximal portion 66 of the housing 30 and is configured to engage a distal end of the elongate body 14 to couple the reload assembly 18 to the elongate body 14. In certain aspects of the disclosure, the retaining member 72 includes projections 78 that are received in openings 80 in the proximal portion 66 of the housing 30 of the reload assembly 18 to secure the retaining member 72 and the coupling member 74 to the housing 30. It is envisioned that other coupling mechanisms can be used to secure the reload assembly 18 to the elongate body 14.

FIG. 3 illustrates the anvil assembly 20 supported on the reload assembly 18 in the clamped position. The anvil assembly 20 includes an anvil head assembly 84 and an anvil center rod assembly 86. In some aspects of the disclosure, the anvil head assembly 84 includes an annular anvil plate 87 and is supported on the anvil center rod assembly 86 about a pivot member 88 such that the anvil head assembly 84 can pivot about the pivot member 88 between operative and tilted positions. For a more detailed description of an anvil assembly including a pivotable head assembly, see, e.g., U.S. Pat. No. 6,945,444 (“the '444 Patent”).

In certain aspects of the disclosure, the approximation mechanism 100 of the surgical stapling device 10 includes an anvil retainer 102 having a trocar 104 that is configured to be releasably coupled to the anvil center rod assembly 86 to couple the anvil assembly 20 to the approximation mechanism 100. In aspects of the disclosure, the approximation mechanism 100 includes a flexible threaded shaft 104 that is received within a threaded bore 105 defined by a proximal portion of the anvil retainer 102. The threaded shaft 104 is rotated via actuation of the buttons 26 (FIG. 1) on the handle assembly 12 to move the anvil assembly 20 in relation to the reload assembly 18 in the direction indicated by arrows “A” in FIG. 3 to move the tool assembly 16 between the open and clamped positions. For a detailed description of an exemplary anvil retainer 102, see the “444 Patent. The inner housing portion 54 of the housing 30 defines a through bore 110 that receives the anvil retainer 102 and the center rod assembly 86 to facilitate movement of the tool assembly 16 between the advanced and retracted positions.

The reload assembly 18 includes a first motor 120 for advancing the annular pusher 46 and staple pushing member 44 from their retracted positions to their advanced positions, and a second motor 122 for advancing the knife carrier 36 and the annular knife 38 from their retracted positions to their advanced positions. In certain aspects of the disclosure, the first and second motors 120, 122, respectively, are piezo electric transducers that expand in response to application of electric current to the motor, e.g., energization. Alternately, other micro actuators capable of advancing the knife carrier 36 and the annular pusher 46 could be used as the first and second motors, e.g., micro-electromechanical systems (MEMS), magnetic motors including solenoids, etc.

The first motor 120 is supported within the housing 30 of the reload assembly 18 and includes a proximal face 124 that is engaged with a shoulder 126 of the housing 30. The first motor 120 is coupled to a power source, e.g., batteries in the handle assembly 12, by a conductor, e.g., a wire 130 that extends from the handle assembly 12 through the elongate body 14. When current is supplied to the first motor 120, the first motor 120 expands longitudinally. Since the proximal face 124 of the first motor 120 is engaged with the shoulder 126 of the housing 30, the first motor 120 expands distally within the housing 30 to advance the annular pusher 46 and the staple pushing member 44 distally within the housing 30 to eject staples “S” from the staple cartridge 32. The wire 130 is coupled to battery pack 24a (FIG. 1) supported within the stationary grip 24 of the handle assembly 12. In certain aspects of the disclosure, a distal portion of the first motor 120 is coupled to a proximal portion of the annular pusher 46 such that the annular pusher 46 is returned to its retracted position when the first motor 120 is deenergized.

Similarly, the second motor 122 is supported within the housing 30 of the reload assembly 18 and includes a proximal face 130 that is engaged with a shoulder 132 supported on the inner housing portion 54 of the housing 30. The second motor 122 is coupled to the battery pack 24a (FIG. 1) in the handle assembly 12 by a conductor, e.g., a wire 140 that extends from the handle assembly 12 and through the elongate body 14. When current is supplied to the second motor 122, the second motor 120 expands longitudinally. Since the proximal face 130 of the second motor 120 is engaged with the shoulder 132 of the inner housing portion 54, the second motor 122 expands distally within the housing 130 to advance the knife carrier 36 and the annular knife 38 distally into the anvil assembly 20 to cut tissue. In certain aspects of the disclosure, a distal portion of the second motor 122 is coupled to the knife carrier 36 such that the knife carrier 36 is returned to the retracted position when the second motor 122 is deenergized.

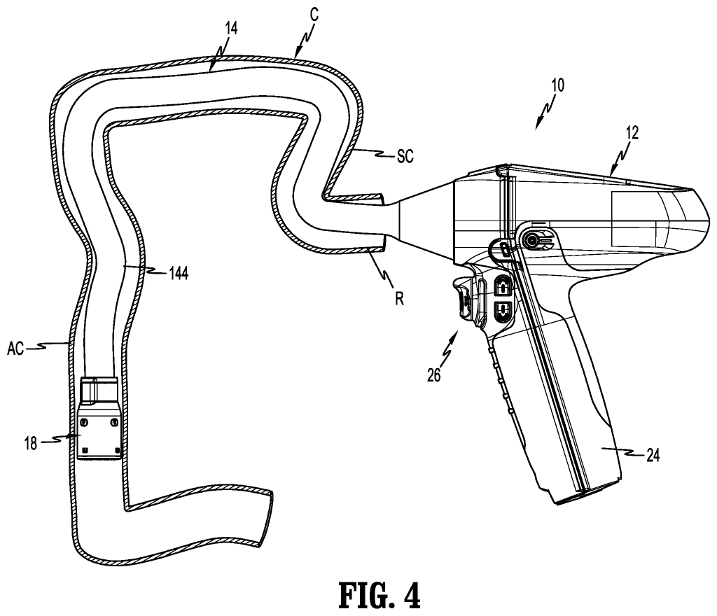

FIG. 4 illustrates the elongate body 14 of the stapling device 10 positioned deep within a body organ, e.g., the colon “C”. The elongate body 14 of the stapling device 10 includes a flexible outer tube 144 that encloses the approximation mechanism 100 of the stapling device 10. Since the reload assembly 18 includes first and second motors 120, 122, respectively, mounted within the housing 30, the stapling device 10 does not have a firing mechanism or a knife advancement mechanism supported within the elongate body 14. As such the elongate body 14 is easily bendable and can be snaked through the colon “C”. As illustrated, the elongate body 14 is inserted through the rectum “R” and advanced through the sigmoid colon “SC”, through the transverse colon “TC”, and into the Ascending cecum or colon “AC”.

FIG. 5 illustrates the stapling device 10 in the clamped position as the stapling device 10 is actuated to fire staple “S”. The button 26 (FIG. 1) on the handle assembly 12 associated with the first motor 120 is actuated to energize the first motor 120. When the first motor 120 is energized, the first motor 120 expands in the longitudinal direction indicated by arrows “B” to advance the annular pusher 46 within the housing 30 of the reload assembly 18 in the direction of arrows “C”. The annular pusher 46 is engaged with the staple pushing member 44 such that advancement of the annular pusher 46 causes corresponding advancement of the staple pushing member 44. As the staple pushing member 44 is advanced, the fingers 50 of the staple pushing member 44 translate through the staple pockets 40 of the staple cartridge 32 to eject the staples “S” into the anvil head assembly 84 of the anvil assembly 20.

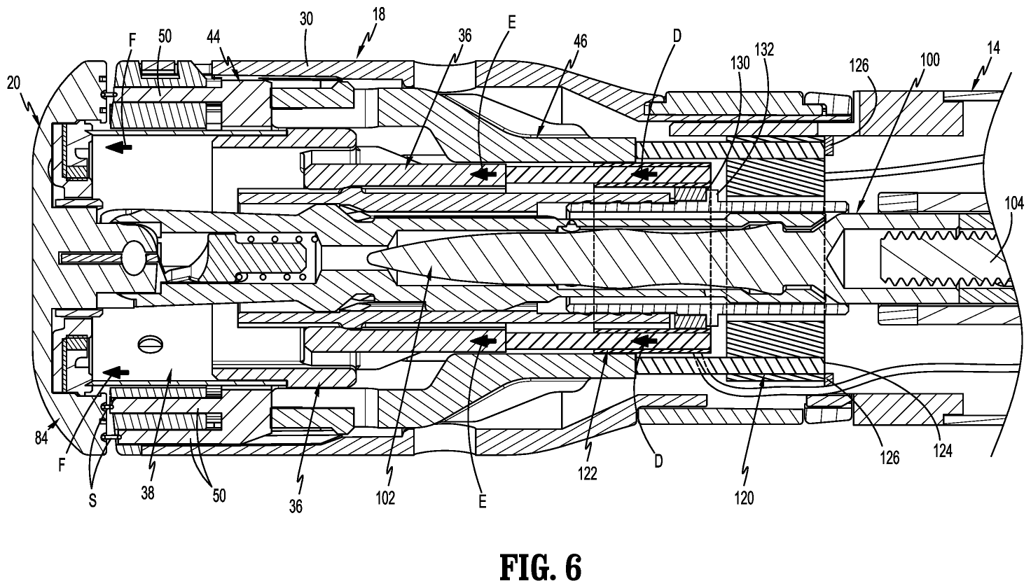

FIG. 6 illustrates the stapling device 10 as the annular knife 38 is advanced to cut tissue. After the staples “S” are fired from the staple cartridge 32 of the stapling device 10, the button 26 (FIG. 1) on the handle assembly 12 associated with the second motor 122 is actuated to energize the second motor 122. When the second motor 122 is energized, the second motor 122 expands in the longitudinal direction indicated by arrows “D” to advance the knife carrier 36 within the housing 30 of the reload assembly 18 in the direction of arrows “E”. The knife carrier 36 supports the annular knife 38 to advance the annular knife 38 in the direction of arrows “F” into the anvil head assembly 84 of the anvil assembly 20 to cut tissue clamped between the staple cartridge 32 of the reload assembly 18 and the anvil head assembly 84 of the anvil assembly 20.

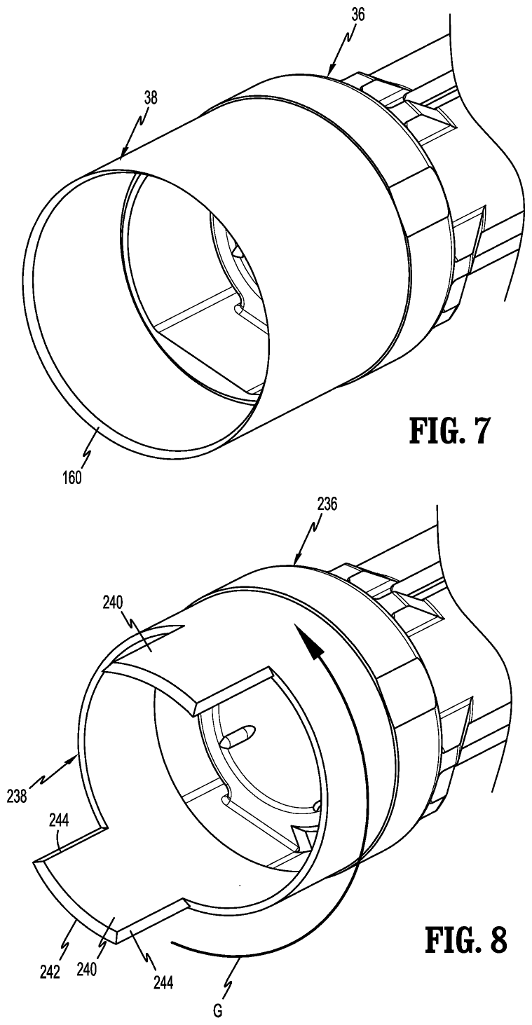

The annular knife 38 (FIG. 7) includes an annular cutting edge 160. As the annular knife 38 is advanced distally into the anvil assembly 20, the cutting edge 160 is pushed into a slices through tissue clamped between the staple cartridge 32 of the reload assembly 18 and the anvil head assembly 84. In another aspect of the disclosure, the second motor 122 (FIG. 2) can be adapted to provide a longitudinal and rotating component to the knife blade 38. In such a device, the knife blade, shown as knife blade 238 in FIG. 8, can include one or more arcuate blade sections 240. Each of the arcuate blade sections 240 includes a distal cutting edge 242 and side cutting edges 244. When the second motor 122 (FIG. 2) is actuated to advance the knife carrier 236 and corresponding advancement of the annular knife 238, the knife carrier 238 is initially advanced longitudinally to advance the arcuate blade section 240 of the annular knife 238 through the tissue and subsequently actuated to rotate the annular knife 238 through the tissue. As the annular knife 238 is advanced longitudinally, the distal cutting edge slices through the tissue. When the annular knife 238 is rotated, the side cutting edges 244 of the arcuate blade sections 240 cut through the tissue as the knife blade is rotated in the direction indicated by arrow “G”. Although two arcuate blade sections 240 are shown spaced 180 degrees apart, it is envisioned that the knife blade 238 can include one or more blade sections that are spaced at a number of angular positions about the knife blade.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A surgical stapling device comprising:
a handle assembly;
an elongate body including a flexible outer tube; and
a tool assembly including an anvil assembly and a reload assembly, the reload assembly including a housing, a staple cartridge defining a plurality of staple pockets, a plurality of staples, each of the plurality of staples being supported within one of the plurality of staple pockets, a staple pushing member positioned within the housing, the staple pushing member movable from a retracted position to an advanced position to eject the plurality of staples from the staple cartridge, a knife supported within the housing, the knife movable from a retracted position to an advanced position to cut tissue, a first motor supported within the housing, wherein energization of the first motor moves the staple pushing member from its retracted position to its advanced position, and a second motor supported within the housing, wherein energization of the second motor moves the knife from its retracted position to its advanced position.

2. The surgical stapling device of claim 1, wherein the first and second motors are piezo electric transducers.

3. The surgical stapling device of claim 2, wherein the first and second motors are coupled to actuation buttons in the handle assembly by wires.

4. The surgical stapling device of claim 3, wherein the handle assembly supports a battery pack.

5. The surgical stapling device of claim 1, wherein anvil assembly includes an anvil head and an anvil center rod, the anvil head supporting an anvil plate.

6. The surgical stapling device of claim 5, wherein the staple cartridge and the anvil plate have an annular configuration.

7. The surgical stapling device of claim 1, further including an approximation assembly having an anvil retainer, the anvil retainer releasably coupled to the anvil assembly and movable from an advanced position to a retracted position to move the tool assembly from an open position to a clamped position.

8. The surgical stapling device of claim 7, wherein the approximation assembly extends from the handle assembly through the elongate body to the tool assembly.

9. The surgical stapling device of claim 1, further including a knife carrier supported within the housing and supporting the knife, the knife carrier movable from a retracted position to an advanced position to move the knife from its retracted position to its advanced position.

10. The surgical stapling device of claim 9, wherein the knife carrier is coupled to the second motor such that when the second motor is deenergized, the knife carrier and the knife are returned to their retracted positions.

11. The surgical stapling device of claim 1, wherein the reload assembly includes an annular pusher supported within the housing, the annular pusher in abutting relation with the staple pushing member and movable from a retracted position to an advanced position in response to energization of the first motor to move the staple pushing member from its retracted position to its advanced position.

12. The surgical stapling device of claim 11, wherein the annular pusher is coupled to the second motor such that when the second motor is deenergized, the annular pusher is returned to its retracted positions.

13. A reload assembly comprising:
a housing, a staple cartridge defining a plurality of staple pockets, a plurality of staples, each of the plurality of staples being supported within one of the plurality of staple pockets, a staple pushing member positioned within the housing, the staple pushing member movable from a retracted position to an advanced position to eject the plurality of staples from the staple cartridge, a knife supported within the housing, the knife movable from a retracted position to an advanced position to cut tissue, a first motor supported within the housing, wherein energization of the first motor moves the staple pushing member from its retracted position to its advanced position, and a second motor supported within the housing, wherein energization of the second motor moves the knife from its retracted position to its advanced position.

14. The reload assembly of claim 13, wherein the first and second motors are piezo electric transducers.

15. The reload assembly of claim 13, further including a knife carrier supported within the housing and supporting the knife, the knife carrier movable from a retracted position to an advanced position to move the knife from its retracted position to its advanced position.

16. The reload assembly of claim 15, wherein the knife carrier is coupled to the second motor such that when the second motor is deenergized, the knife carrier and the knife are returned to their retracted positions.

17. The reload assembly of claim 13, wherein the reload assembly includes an annular pusher supported within the housing, the annular pusher in abutting relation with the staple pushing member and movable from a retracted position to an advanced position in response to energization of the first motor to move the staple pushing member from its retracted position to its advanced position.

18. The reload assembly of claim 17, wherein the annular pusher is coupled to the second motor such that when the second motor is deenergized, the annular pusher is returned to its retracted positions.

19. A surgical stapling device comprising:
an elongate body; and
a tool assembly including an anvil assembly and a reload assembly, the reload assembly including a housing, a staple cartridge defining a plurality of staple pockets, a plurality of staples, each of the plurality of staples being supported within one of the plurality of staple pockets, a staple pushing member positioned within the housing, the staple pushing member movable from a retracted position to an advanced position to eject the plurality of staples from the staple cartridge, a knife supported within the housing, the knife movable from a retracted position to an advanced position to cut tissue, a first motor supported within the housing, wherein energization of the first motor moves the staple pushing member from its retracted position to its advanced position, and a second motor supported within the housing, wherein energization of the second motor moves the knife from its retracted position to its advanced position.

20. The surgical stapling device of claim 19, wherein the elongate body includes a flexible outer tube and the first and second motors are piezo electric transducers.
